(19) United States
(12) Patent Application Publication
(10) Pub. No.: US 2009/0069877 A1
(43) Pub. Date: Mar. 12, 2009

(54) ELECTRODE DEVICE FOR PHYSIOLOGICAL USE, IN PARTICULAR IN CARDIOLOGY

(76) Inventors: Gernot Kolberg, (US); Erhard Flach, (US); Marc Schurr, (US); Stephan Zedler, (US)

Correspondence Address: DALINA LAW GROUP, P.C., 7910 IVANHOE AVE. #325, LA JOLLA, CA, 92037, US

(21) Appl. No.: 12/206,918

(22) Filed: Sep. 9, 2008

(30) Foreign Application Priority Data
Sep. 10, 2007 (DE) 10 2007 043 127.0

(51) Int. Cl. A61N 1/04

(52) U.S. Cl. 607/121

(57) ABSTRACT

An electrode device for physiological use, in particular in cardiology, that has an electrically insulating, elastic electrode body which, between internal and external layers (1, 2), has an intermediate layer (3) tailored to their physical properties.

This application takes priority from German Patent Application DE 10 2007 043 127.0, filed 10 Sep. 2007, the specification of which is hereby incorporated herein by reference.

### BACKGROUND OF THE INVENTION

1. Field of the Invention

The invention relates to an electrode device for physiological use, in particular in cardiology, having an electrode body constructed in layers from electrically insulating elastic materials.

2. Description of the Related Art

It is to be noted on the background of the invention that various electrode properties, such as electrical insulation, mechanical properties such as bending and torsion rigidity, friction properties, and adequate abrasion protection are relevant for a practical electrode body which is used in coronary vessels as a signal generator of implanted cardiac pacemakers, for example. These various properties place differing and partially contradictory requirements on the materials used for the electrode body, as is clear from the following practical example. Thus, electrode bodies are typically produced for physiological reasons from silicone, which has very long-term chemical and electrical stability. However, this material has the disadvantage that it has poor friction properties and displays a high risk of abrasion.

Applying a thin friction layer to the silicone to improve the friction properties is known in this context from the prior art. On the other hand, a polyurethane layer may be provided on the silicone body to improve the abrasion properties—and to a certain extent also the friction properties. “Layer” is to be understood to mean—which is to apply throughout for the further disclosure of the invention—a separate hose, an envelope, or a coating applied to a supporting layer part.

The disadvantage of the existing approaches to achieving the object of improving the friction and abrasion properties is that the combination of the specified materials causes mechanical tensions at the interface to the particular other material because of their differing properties, which may assume such an extent that in the long-term use of the electrode device, at least one of the two material partners is damaged. Thus, a very thin, hard friction coating, generated by fluorination, of a silicone insulation hose of the electrode body may obtain very fine surface cracks which propagate in the silicone material and may thus decrease the tensile strength of the silicone.

In the event of a material pairing of polyurethane on silicone, the adhesion between these two material partners may be lost upon long-term use because of the tensions between the materials. In the event of a continuous movement of the electrode device, as may be the case in an electrode implanted in a coronary vessel and floating in the bloodstream, for example, this results in friction between the material partners and thus abrasion of the silicone. Fundamental causes for these disadvantages, which may become a problem in particular in long-term use of an electrode device, are the tensions occurring between the various materials.

### BRIEF SUMMARY OF THE INVENTION

Proceeding from the described problems of the prior art, the invention is based on the object of implementing an electrode body constructed in layers from electrically insulating, elastic materials in such a way that the electrode devices equipped therewith have particular long-term stability in physiological use.

This object is achieved by the features specified in the characterizing part of Claim 1, according to which
a gradient of the values of a physiological property of the layers is provided between an internal layer and an external layer of the electrode body, and
at least one intermediate layer, which has at least one intermediate value of these physical properties lying between the values of the external and internal layers, is used between internal layer and external layer.

The invention is thus based on the conceptual idea of reducing the tensions in the interfaces caused by the differing values of a physical property of the internal and external layers, such as a differing hardness or elasticity, for example, in that the materials of differing properties are separated by materials whose properties lie between them. I.e., a gradient of the values of a physical property, such as the hardness, is provided between the internal and external layers, which is “ameliorated” by the intermediate layer used between them. In other words, the gradient of the values of the physical properties of the internal and external layers does not change due to the intermediate layer, but the intermediate layer causes the “large” jump between the values of the physical properties of the internal and external layers to be divided into at least two “small” jumps. The latter has an intermediate value of the particular physical property lying between the values of the internal and external layers. The corresponding physical variable then does not complete a single jump, but rather two or more steps or a gradient, upon the transition between the layers. The tensions in the interfaces between the layers are thus decreased and the problems described on the prior art are at least significantly reduced. Further problems, such as appearances of detachment, deformation—for example, in the form of wrinkling—upon bending of the electrode body, or internal friction of the electrode body may thus also be reduced.

### BRIEF DESCRIPTION OF THE DRAWINGS

Preferred embodiments of the invention result from the dependent claims. Their features, details, and advantages are also explained in greater detail in the following description on the basis of the appended drawing.

FIG. 1 shows a schematic section through an electrode body.

### DETAILED DESCRIPTION OF THE INVENTION

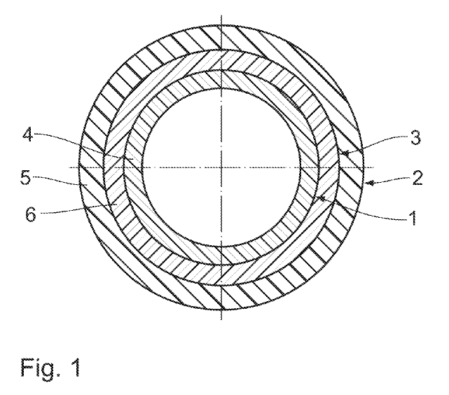

As is clear from FIG. 1, the electrode body has an internal layer 1 and an external layer 2. An intermediate layer 3 is placed between them. In the embodiment shown, all layers 1, 2, and 3 comprise hoses 4, 5, and 6. The hose 4 forming the internal layer 1 may be made of soft silicone, the hose 5 forming the external layer 2 of hard polyurethane, and the hose 6 forming the intermediate layer 3 may be a moderate-hardness polyurethane hose. The hoses 4 through 6 may lie closely on one another, a lubricant being able to be introduced between them instead of air. Gluing the hoses 4 through 6 to one another is also possible depending on the intended application.

The internal layer 1 may also be formed from copolymers, for example. The external layer 2 may preferably be formed from PTFE, especially preferably as Teflon®, or copolymers, especially preferably from silicone-urethane copolymers. In a further variant, a primer is used as the intermediate layer 3 between the internal layer 1 and the external layer 2. This primer is advantageous if coatings are to be used instead of hoses.

Finally, coatings having different values of the particular properties of the material may also be used for the implementation. The following table provides an exemplary selection for various implementations of the layers 1 through 3 in this context:

| Example | Internal layer 1 | Intermediate layer 2 | External layer 3 |
|---|---|---|---|
| 1 | Soft silicone hose | Soft PU hose | Hard PU hose |
| 2 | Soft silicone hose | Soft PU coating | Hard PU hose |
| 3 | Soft silicone hose | Soft PU hose | Hard PU coating |
| | | (hardness 80 AE) | (hardness 55 D, |
| | | | thickness 20 μm) |
| 4 | Soft silicone hose | Soft PU coating | One or more |
| | | (hardness 80 A) | PU coatings - |
| | | | becoming harder |
| | | | toward the outside |
| | | | (hardness 60 A, |
| | | | 55D) |
| 5 | Soft silicone hose | Adhesive coating | Hard PU coating |
| | | made of copolymer | |

The following remarks are to be made on the above-mentioned examples:

In example 3, the external hard PU coating causes greater risk of degradation than that with a PU hose having a hardness of 80 AE and improves the friction properties.

In example 4, the internal layer 1 may also be formed by a silicone hose which is hard per se, which is provided with a well-adhering soft silicone coating. A well-adhering PU coating which is somewhat less soft and a hard and abrasion-resistant PU external layer are applied thereon.

What is claimed is:

1. An electrode device for physiological use, in particular in cardiology, comprising:
an electrode body constructed in layers from electrically insulating, elastic materials, wherein a gradient of values of a physical property of layers (1, 2) is provided between an internal layer (1) and an external layer (2) of the electrode body, and wherein at least one intermediate layer (3) is used between the internal layer (1) and the external layer (2), wherein said at least one intermediate layer (3) has at least one intermediate value of the physical property lying between values of the physical property of the external and internal layers.

2. The electrode device according to claim 1, wherein the physical property is hardness, elasticity, bending rigidity, or torsional rigidity.

3. The electrode device according to claim 1, wherein layers (1, 2, 3) having different values of physical properties are formed by hoses (4, 5, 6), coatings, or combinations thereof.

4. The electrode device according to claim 1, wherein the internal layer (1) is formed by a soft silicone hose (4) and the intermediate layer (3) and external layer (2) are formed by polyurethane hoses (5, 6) which are harder in steps towards outside of the electrode body and in relation thereto.

5. The electrode device according to claim 1, wherein when the layers (1, 2, 3) are implemented in the form of hoses (4, 5, 6) seated one inside another, the layers are glued to one another.
